(19) United States
(12) Patent Application Publication
(10) Pub. No.: US 2010/0016753 A1
(43) Pub. Date: Jan. 21, 2010

(54) Systems and Methods for Portable Neurofeedback

(76) Inventor: Katrina S. Firlik, New Canaan, CT (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 12/503,403

(22) Filed: Jul. 15, 2009

Related U.S. Application Data
(60) Provisional application No. 61/081,940, filed on Jul. 18, 2008.

(51) Int. Cl. A61B 5/0482

(52) U.S. Cl. 600/545

(57) ABSTRACT

Systems and methods for portable neurofeedback are disclosed herein. In an embodiment, a neurofeedback system includes sensors for capturing, recording and transmitting brain electrical activity; and a portable device for receiving, processing and displaying in real-time the brain electrical activity, wherein the brain electrical activity is provided visually on the portable device as visual feedback. In an embodiment, the portable device further includes speakers for implementing auditory feeedback based on the recorded brain electrical activity. In an embodiment, the portable device further includes at least one vibration feedback motor for implementing vibratory feedback based on the recorded brain electrical activity.

### RELATED APPLICATIONS

This application claims the benefit of and priority to U.S. Provisional Application Ser. No. 61/081,940, filed Jul. 18, 2008, the entirety of this application is hereby incorporated herein by reference.

### FIELD

The embodiments disclosed herein relate to neurotherapy, and more particularly to systems and methods for portable neurofeedback.

### BACKGROUND

Electroencephalography is the neurophysiologic measurement of the electrical activity of the brain by recording from electrodes placed on the scalp or, in special cases, on the cortex. The resulting traces are known as an electroencephalogram (EEG) and represent so-called brainwaves. The bioelectrical profiles of the EEG brainwaves are composed of information on thought, metabolic state, hormonal state, attention, feelings and emotions. With the help of neurotransmitters, EEG brainwaves communicate to direct movement, sleep, thought and attention.

Neurofeedback (also known as EEG biofeedback and neurotherapy) is a therapy technique that presents a user with real-time feedback on brainwave activity, and can provide cognitive, emotional and clinical benefits by teaching the user to manage, control and manipulate brainwave activity. In some circumstances, neurofeedback has been shown to enhance attention (concentration, ability to focus), which can facilitate improvement in memory, learning, behavior, self-esteem, and general “brain fitness.” Such benefits can be widely helpful across almost any discipline in achieving “peak performance” and include, for example, academics, sports, career pursuits, performing arts, and personal relationships. For example, in sports, the ability to focus and tune out distractions can maximize both mental and physical abilities during a game. Similarly, neurofeedback has been used for treating some common conditions and disorders. Sometimes the brain transitions into inappropriate states, resulting in the mis-direction of emotions or actions. These inappropriate brain states may lead to a number of conditions and disorders in people including, but not limited to, attention-deficit/hyperactivity disorder (ADHD), migraines, seizures, sleep disorders, pre-menstrual disorder, extreme stress, anxiety attacks, autism, depression and learning disabilities. It is believed that with the aid of neurofeedback, a user can train and control their brain to stop these inappropriate states from manifesting.

Recently, neurofeedback is being advocated for use in the general population as an adjunct to what is broadly referred to as brain fitness or meditation. Similar to meditation, neurofeedback can be used to enhance attention, concentration, and the ability to focus. Unlike meditation however, neurofeedback provides objective visual or other sensory cues as to a user's brain state. Neurofeedback can therefore be considered a form of instrumented meditation. Further, unlike meditation, which can produce positive results after months or even years, neurofeedback can often achieve the same or similar results in much less time.

Although the potential applications of neurofeedback are vast, it has been underutilized thus far for a variety of reasons. Neurofeedback suffers from a fringe status in mainstream medicine (partly because the first line treatment for the same disorders are pharmaceuticals, and partly because a lack of research funds have precluded large, well designed, well executed studies). In addition, neurofeedback typically requires a skilled therapist to administer, typically in an office setting over numerous sessions, which limits its flexibility and practicality.

### SUMMARY

Systems and methods for portable neurofeedback are disclosed herein.

According to aspects illustrated herein, there is provided a neurofeedback system that can be used by an “ordinary” person (i.e., does not have to be a skilled therapist), that is portable, that provides real-time feedback, and that is easy-to-use. The neurofeedback system includes sensors for capturing, recording and transmitting brain electrical activity; and a portable device for receiving, processing and displaying in real-time the brain electrical activity, wherein the brain electrical activity is provided visually on the portable device as visual feedback. In an embodiment, the portable device further includes speakers for implementing auditory feedback based on the recorded brain electrical activity. In an embodiment, the portable device further includes at least one vibration feedback motor for implementing vibratory feedback based on the recorded brain electrical activity. In an embodiment, the portable device further includes at least one vibration feedback motor for implementing rumble feedback based on the recorded brain electrical activity.

According to aspects illustrated herein, there is provided a method for neurotherapy that includes providing a neurofeedback system to a user, wherein the neurofeedback system includes sensors for capturing, recording and transmitting brain electrical activity and a portable device for receiving, processing and displaying in real-time the brain electrical activity; configuring the portable device to select a neurofeedback program, wherein the neurofeedback program is loaded within the portable device; placing the sensors on the user such that the brain electrical activity of the user can be captured, recorded and transmitted to the portable device; executing the neurofeedback program on the portable device, wherein the real-time brain electrical activity of the user is continuously displayed on the portable device as visual feedback and the user is able to control the brain electrical activity such that the visual feedback can be manipulated by the user in real-time; and completing the neurofeedback program.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a block diagram showing an embodiment of a neurofeedback system of the present disclosure. The neurofeedback system includes a portable device in communication with sensors. The sensors capture, record, and transmit brain electrical activity from a user to the portable device. The portable device receives, processes, and displays in real-time the brain electrical activity of the user, giving the user visual and/or or other sensory cues on states of emotion and cognition of the user.

FIG. 2 is a block diagram showing some of the main components of the portable device of FIG. 1 in communication with the sensors of FIG. 1.

FIG. 3 is a flow diagram showing an embodiment of a method of neurotherapy using the neurofeedback system of FIG. 1.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Researchers in the 1930's and 1940's identified 4 categories of brainwaves, each representing a different level of the brain's activity. The first category of brainwaves is beta (which oscillates at about 13 to about 38 Hz). When the brain is actively engaged in an activity, beta brainwaves are being displayed. The second category of brainwaves is alpha (about 8 to about 13 Hz). Where beta represents arousal, alpha represents the opposite—non-arousal. For example, an alpha state may occur after someone just completes a task and sits down to rest. Certain forms of meditation or taking a “time out” to relax and reflect would also put someone in an alpha state. The third category of brainwaves is theta (about 4 to about 7 Hz). This state can occur when someone is performing a task that is so automatic that they can mentally disengage from the task—a repetitious activity like brushing hair, or a monotonous one, like driving on a sparsely populated highway. Similarly, going on a morning run can also put someone in a theta state. Delta brainwaves are the slowest in frequency of all four brainwaves (below about 4 Hz). A delta state can be seen when someone is sleeping deeply. “Sleeping deeply” refers to dreamless, non-REM sleep, when the body is healing, restoring, and resetting. Other types of brainwaves have recently been identified, such as the Sensory motor rhythm (SMR, around 14 Hz), which seems to link brain and body functions, and gamma brain waves (39-100 Hz), which are involved in higher mental activity and consolidation of information.

A person does not ever produce only “one” brain wave type. Overall brain activity is a mix of all the frequencies at the same time, some in greater quantities and strength than others. Balance of the brainwaves is important for optimal functioning, and therefore, the brain is constantly shifting brain activity during the day to match what a person is doing. Therefore, flexibility is a key goal for efficient brain functioning. Resilience is important for the brain to be and stay effective. When something goes wrong, likely it is because the brain is lacking either flexibility or resilience. Therefore, the brain needs to be both flexible—able to adjust to whatever one is wanting to do—and resilient—able to go with the flow. To do this, the brain needs access to a variety of different brain states. These states are produced by different patterns and types of brain wave frequencies.

Neurofeedback is a method for increasing both flexibility and resilience of the brain by using the EEG to see a users brainwaves. Neurofeedback trains the behavior of brainwaves, and does not try to promote one type of specific activity over another. With neurofeedback, software is the component that gives the user the visual feedback on states of emotion and cognition that are extracted from a real-time EEG recording. The brain then learns to improve the management of these states. Once these new developmental skills are learned, they typically become automatic. A person can make use of these different brain states to develop certain skills, such as using the theta state to plan the day's activities or using the delta state to enhance language retention. For example, if a person wants to feel less stress and anxiety, improve the strength of their immune system, be more creative, and have more peak performance in athletic activity, as well as in other areas of one's life, it may be possible to use neurofeedback to try to increase the amount of one's alpha brainwaves.

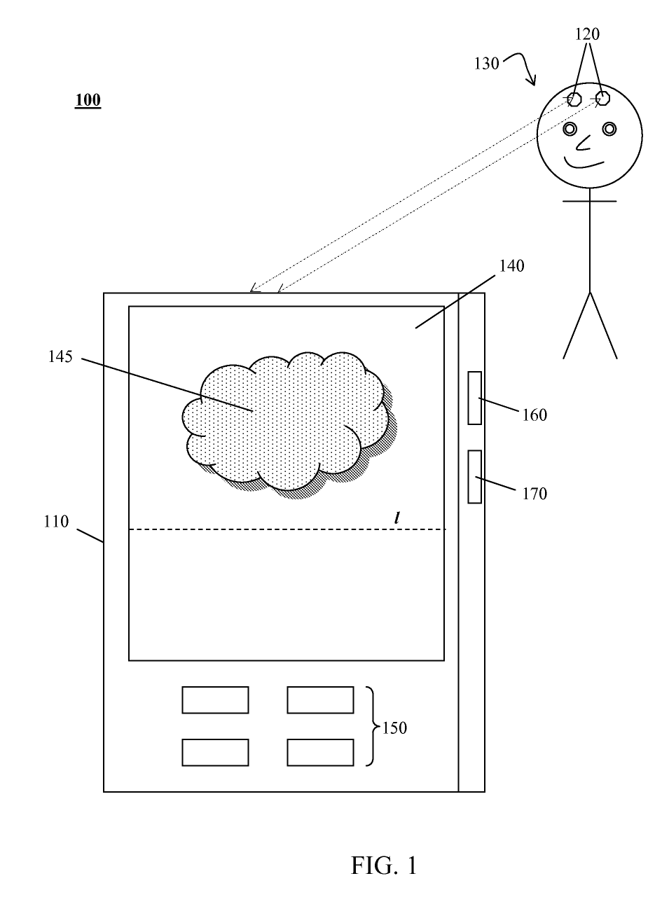

FIG. 1 is a block diagram showing an embodiment of a neurofeedback system 100 of the present disclosure. The system 100 includes a portable device 110 in communication with sensors 120 placed on a user 130 of the portable device 110. The sensors 120 pick up a wide variety of signals or brainwaves from the user 130. The sensors 120 are in communication with the portable device 110, which includes all the major components needed to conduct neurotherapy in a simple, easy-to-use way. As used herein, the term “portable” refers to a wearable or handheld device. The portable device 110 allows the user 130 to conduct neurofeedback whilst being mobile. Therefore, the portable device 110 is designed to be moved from one place to another, and is easily transportable. The portable device 110 may, for example, be easily carried in a handbag, in a gym bag, in a briefcase, in a piece of luggage, or in a pocket of an article of clothing. The sensors 120 may transmit signals to the portable device 110 via one or a plurality of frequency channels and supports a frequency range from about 0.25 to about 100 Hz (cycles/second). In an embodiment, the frequency range is from about 1 to about 40 Hz. In an embodiment, the frequency range is from about 1 to about 20 Hz. The portable device 110 allows for the use of neurofeedback in a more personalized, portable, user-friendly way. The portable device 110 can be used in an office setting, at home, at a gym, at an airport, in the car, etc. The portable device 110 may be, for example, a Personal Digital Assistant (PDA), a cell phone, for example an iPhone® an N-Gage, and a Blackberry®, a handheld gaming device, for example a Nintendo Game Boy, a Nintendo DS, a POSBRO G100 Mobile WiMAX, Sony PlayStation Portable, or similar electronic devices, and a computer, for example a laptop and a netbook.

The portable device 110 includes an on/off control 160 as well as one or more function buttons 150. The function buttons 150 provide controls for the portable device 110. For example, one function button may be to capture an EEG baseline signal from the user 130 prior to the start of a neurofeedback session, another function button may be to begin the neurofeedback session, and one or more other function button(s) may enable the selection of a graphic display to display the real-time feedback of the user 130. In an embodiment, there is also a volume control 170. The controls may be for example, buttons or a sliding wheel. The signals picked-up by the sensors 120 are filtered and processed by the portable device 110, and feedback is provided visually (visual feedback) on a display screen 140. In an embodiment, the display screen 140 is a liquid crystal display (LCD) screen. The display screen 140 is a screen that can show a visual representation 145 of the device user's 130 brainwave patterns. The visual representation 145 may take any number of forms, such as moving graphs of various colors and peaks and valleys, one or more concrete or abstract images, brain waveforms, video clips, etc. In an embodiment, the portable device 110 includes speakers such that auditory feedback, as well as visual feedback, is possible. In such an embodiment, the portable device 110 can include an auditory feedback tool or software module that is separate or part of the software program of the portable device 110. In an embodiment, the neurofeedback system includes a vibration feedback motor for implementing vibratory feedback. In an embodiment, the portable device further includes at least one vibration feedback motor for implementing rumble feedback based on the recorded brain electrical activity. In such embodiments, the portable device 110 can include a software module for executing the vibratory and/or rumble feedback that is separate or part of the software program of the portable device 110. In an embodiment, the various components (e.g., the on/off control 160, the function buttons 150, the volume control 170, and the display screen 140) of the portable device 110 are configured as illustrated in FIG. 1. In an embodiment, the various components (e.g., the on/off control 160, the function buttons 150, the volume control 170, and the display screen 140) of the portable device 110 are configured in a different layout then that represented by FIG. 1.

The sensors 120 are electrodes attached to the user 130 in order to read the user's 130 brainwaves. In an embodiment, the sensors 120 are attached to the user's 130 scalp/head. The sensors 120 may be attached to the scalp/head of the user 130 via various means, including, but not limited to, adhesives, headbands, visors, caps/hats, helmets, clamps, clips and earring studs. The sensors 120 may be disposable or re-usable. Although FIG. 1 shows the portable device 110 in communication with two sensors 120, it should be noted that any number of sensors 120 can be used in conjunction with the portable device 110. Also, the sensors 120 can communicate with the portable device 110 wirelessly or via a wire.

In an embodiment, the portable device 110 includes memory. The portable device 110 can utilize memory to store the user's 130 neurofeedback session parameters, including the user's 130 progress, such as the length of time needed for the user 130 to obtain a focused state and/or to the length of time that the user 130 remains in the focused state. The portable device 110 can then analyze the stored information, such as to determine whether the user 130 is improving each time the user 130 uses the portable device 110.

In an embodiment, the neurofeedback system 100 of the present disclosure provides at least one of cognitive, emotional and clinical benefits. For example, the neurofeedback system 100 may be used to enhance attention (concentration, ability to focus), which can facilitate improvement in memory, learning, and general “brain fitness.” The disclosed neurofeedback system 100 may help restore and improve a user's 130 ability to focus more quickly, more completely, and for greater lengths of time.

The neurofeedback system 100 may be used as a form of instrumented meditation, for example, to help the user 130 via strengthening of the power of the mind, to induce a state of calm awareness and to achieve a greater emotional stability and resilience. Happiness and satisfaction are largely internally mediated, and techniques such as neurofeedback allow the user 130 to achieve greater control over internal states. Because the neurofeedback system 100 allows for an instantaneous reading and visual feedback of mental state, neurofeedback can potentially be more effective than meditation over a shorter time course.

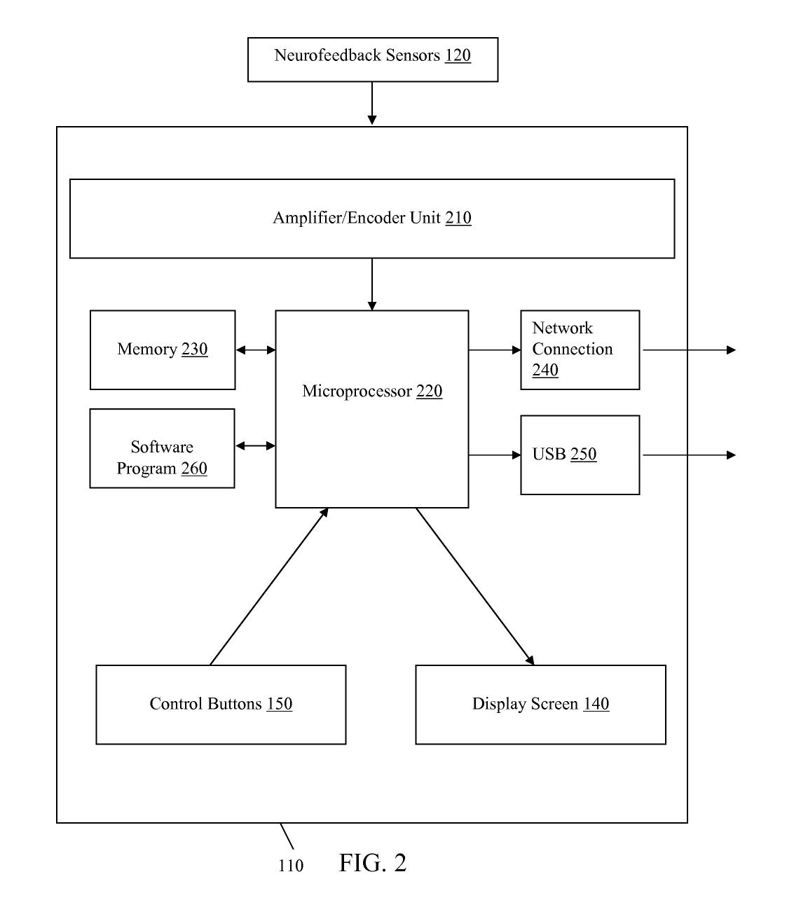

Referring to FIG. 2, in an embodiment the portable device 110 includes an amplifier/encoder unit 210, a microprocessor 220, memory 230, a network connection 240, a universal serial bus (USB) connector 250, the function buttons 150 and the display screen 140. The sensors 120 are used to capture and record brain electrical activity (EEG signals) from the user 130. In an embodiment, the sensors 120 transmit (wirelessly or over a wire) the EEG signals representing the brain electrical activity to the amplifier/encoder unit 210. This transmission may occur continuously, periodically, a set number of times, etc. The amplifier/encoder unit 210 magnifies and converts the EEG signals from analog to digital format and sends the digitized EEG signals to the microprocessor 220 for processing the brain activity associated with the user 130. The microprocessor 220 receives the digitized signals and with the help of a software program 260, processes the digitized signals in order to display, in real time, the visual representation 145 on the display screen 140, representing the brain activity of the user 130. In an embodiment, the EEG signals are taken about 120 times per second and digitized. In an embodiment, the EEG signals are taken about 256 times per second and digitized. In an embodiment, the EEG signals are taken about 500 times per second and digitized. In an embodiment, the EEG signals are taken about 1000 times per second and digitized. Various known biofeedback software programs may be used in conjunction with the portable device 110, including, but not limited to, BioExplorer, BrainMaster, BioGraph Infiniti, BioEra, BrainPaint and similar programs. Alternately, a software program created specifically for the portable device 110 may be used for a specific application of the neurofeedback system 100 of the present disclosure.

The microprocessor 220 may also be connected to memory 230. Memory 230 may include read-only memory (ROM) (e.g., EPROM, EEPROM, and/or flash memory) and/or random access memory (RAM), such as dynamic RAM (DRAM).

As described above, the display screen 140 is a screen that can show the visual representation 145 of the device user's 130 brainwaves. In an embodiment, the visual representation 145 shown on the display screen 140 includes peaks and valleys that will vary in height and/or in width. These pattern variations become the visual feedback that allows the user 130 to learn how to control the production of the user's 130 brainwaves. The visual representation 145 may take any number of forms, such as one or more images, movies, brain waveforms, etc. In an embodiment, EEG signals that are correlated with a first state, such as a relaxed state or a state indicating that the user 130 is paying attention, are rewarded with one or more particular images being displayed on the display screen 140. EEG signals associated with a second state, such as a tense state or a distracted state, are not rewarded. For example, in FIG. 1 the display screen 140 displays the visual image representation of a cloud 145 that has moved above a line “l” indicating that the user 130 is in the first state. If the cloud 145 moves below the line “l”, the user 130 is in the tense second state. In another example, the display screen 140 displays an image of a waterfall when the neurofeedback associated with the user 130 indicates that the user 130 is in the relaxed state. Alternatively, the portable device 110 may display an image of lightning when the neurofeedback associated with the user 130 indicates that the user 130 is in the tense state. Another example is the portable device 110 displaying an image of a car that remains stationary if the user 130 is distracted. The car can move forward if the user 130 is able to focus and pay attention to the car and the user's 130 ability to move the car. Further, the car may speed up with improvement in focus. In another embodiment, the image displayed by the portable device 110 can be personalized to the individual interests of the user 130. In another embodiment, the portable device 110 outputs an audio sound representing the user's 130 brainwaves. In an embodiment, the portable device 110 outputs a vibratory sensation representing the user's 130 brainwaves. It should be understood that the portable device 110 is capable of providing real-time neurofeedback to the user 130 through various forms and combinations of sensory cues.

In an embodiment, the portable device 110 includes a network connection 240 which enables connection to a network such as the Internet. As a result, in an embodiment the user 130 can transmit his or her results to his or her doctor, or to various websites, for further review and analysis. The portable device 110 may also include a universal serial bus (USB) connector so that the portable device 110 can connect to another device, such as a personal computer, although connection to a personal computer is not necessary.

As described above, the portable device 110 also includes function buttons 150. The function buttons 150 enable the user 130 to edit settings of the portable device 110. For example, the portable device 110 may include several neurofeedback programs that a user 130 can choose from using the function buttons 150.

Although the portable device 110 is shown with a plurality of components, it should be noted that one or more of the components shown in FIG. 2 may be in communication with but located external to the portable device 110. For example, the portable device 110 may communicate with additional memory that is in communication with the portable device 110 but not internal to the portable device 110. Similarly, the amplifier/encoder unit 210 may be located external to the portable device 110.

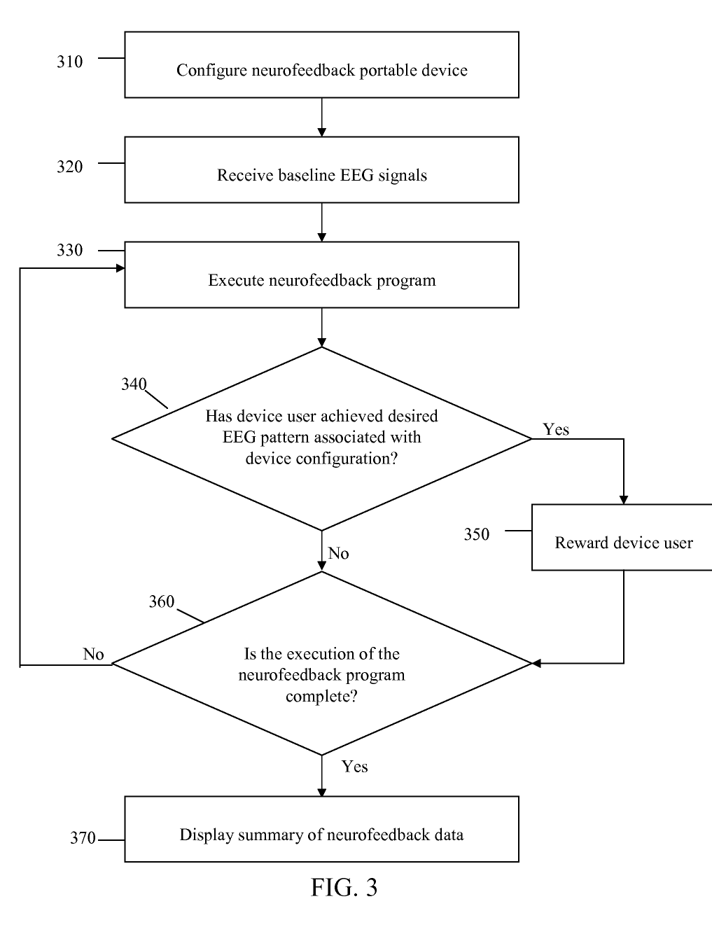

FIG. 3 shows an embodiment of the steps performed by the user 130 of the neurofeedback system 100 during a neurotherapy session. In step 310, the user 130 of the portable device 110 configures the portable device 110 using the function buttons 150. For example, the user 130 may select one of a plurality of neurofeedback sessions to execute (such as if the user 130 wants to work on improving attention span, the user 130 can select a neurofeedback program used to facilitate improvement in attention span). The user 130 may further configure the portable device 110 by adjusting the level of difficulty of the neurofeedback program.

The user 130 then places the sensors 120 (e.g., electrodes) on his or her head. Once this occurs, the portable device 110 can receive baseline EEG signals in step 320. Baseline EEG signals are brain signals of the user 130 when the user 130 first starts using the portable device 110 (i.e., before execution of a neurofeedback program during a neurofeedback session (the time that the sensors 120 are placed on the user's 130 head)).

The portable device 110 then executes the selected neurofeedback program in step 330. In step 340, the portable device 110 determines if the user 130 has achieved a desired EEG pattern (associated with the configuration of the portable device 110 as shown in step 310). A desired EEG pattern represents a desired mental state, such as good concentration.

If the portable device 110 determines that the user 130 has achieved a desired EEG pattern, the portable device 110 rewards the user 130 in step 350. The portable device 110 rewards the user 130 by changing an image on the display screen 140. For example, the portable device 110 can cause an object to move on the display screen 140 when the user 130 achieves the desired EEG pattern. If the user's 130 brain activity is not in the desired EEG pattern, the portable device 110 does not move the object on the display screen 140.

In step 360, the portable device 110 determines whether the execution of the neurofeedback program is complete. If the program's execution is complete, the portable device 110 displays a summary of the neurofeedback data in step 370. This summary of neurofeedback data can include, for example, the time required to achieve the desired mental state and/or the percentage of time in the desired mental state. In an embodiment, the portable device 110 can transmit the data to another computer via its network connection 240.

The neurofeedback systems of the presently disclosed embodiments can be used to treat a wide variety of conditions including, but not limited to, attention-deficit/hyperactivity disorder, chronic pain, acute pain (e.g., post-operative, post-traumatic, and labor pains), anxiety, addiction, learning disabilities and disorders, depression and mood disorders, tourette's syndrome, epilepsy, autism, pervasive developmental disorders, stress, pre-menstrual syndrome, migraines, head injury rehabilitation, as well as improving athletic or artistic performance, among other uses.

A method of neurotherapy includes providing a neurofeedback system to a user, wherein the neurofeedback system includes sensors for capturing, recording and transmitting brain electrical activity and a portable device for receiving, processing and displaying in real-time the brain electrical activity; configuring the portable device to select a neurofeedback program, wherein the neurofeedback program is loaded within the portable device; placing the sensors on the user such that the brain electrical activity of the user can be captured, recorded and transmitted to the portable device; executing the neurofeedback program on the portable device, wherein the real-time brain electrical activity of the user is continuously displayed on the portable device as visual feedback and the user is able to control the brain electrical activity such that the visual feedback can be manipulated by the user in real-time; and completing the neurofeedback program.

All patents, patent applications, and published references cited herein are hereby incorporated by reference in their entirety. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A neurofeedback system comprising:
sensors for capturing, recording and transmitting brain electrical activity; and
a portable device for receiving, processing and displaying in real-time the brain electrical activity, wherein the brain electrical activity is provided visually on the portable device as visual feedback.

2. The neurofeedback system of claim 1 wherein the sensors are electrodes.

3. The neurofeedback system of claim 2 wherein the electrodes transmit the brain electrical activity wirelessly to the portable device.

4. The neurofeedback system of claim 2 wherein the electrodes transmit the brain electrical activity to the portable device via one or a plurality of frequency channels.

5. The neurofeedback system of claim 2 wherein the electrodes support a frequency range from about 0.25 to about 100 Hz.

6. The neurofeedback system of claim 1 wherein the portable device is an iPhone®.

7. The neurofeedback system of claim 1 wherein the portable device includes a display screen for providing the visual feedback.

8. The neurofeedback system of claim 7 wherein the visual feedback indicates one of a first state or a second state of a user of the system.

9. The neurofeedback system of claim 8 wherein the first state represents a focused state of the user.

10. The neurofeedback system of claim 8 wherein the second state represents a distracted state of the user.

11. The neurofeedback system of claim 9 wherein when the user is in the focused state, the visual feedback displayed on the portable device is of a first type.

12. The neurofeedback system of claim 10 wherein when the user is in the distracted state, the visual feedback displayed on the portable device is of a second type.

13. The neurofeedback system of claim 1, wherein the portable device further comprises speakers for implementing auditory feedback based on the recorded brain electrical activity.

14. The neurofeedback system of claim 1, wherein the portable device further comprises at least one vibration feedback motor for implementing vibratory feedback based on the recorded brain electrical activity.

15. The neurofeedback system of claim 1, wherein the system is used to improve a number of conditions and disorders selected from the group consisting of attention-deficit/hyperactivity disorder, chronic pain, acute pain, memory problems, learning disorder, focusing disorder, sleep disorder, tourette's syndrome, pre-menstrual syndrome, migraines, seizures, extreme stress, anxiety attacks, autism and depression.

16. The neurofeedback system of claim 1, wherein the system is used for peak performance training.

17. A method of neurotherapy comprising:
providing a neurofeedback system to a user, wherein the neurofeedback system includes sensors for capturing, recording and transmitting brain electrical activity and a portable device for receiving, processing and displaying in real-time the brain electrical activity;
configuring the portable device to select a neurofeedback program, wherein the neurofeedback program is loaded within the portable device;
placing the sensors on the user such that the brain electrical activity of the user can be captured, recorded and transmitted to the portable device;
executing the neurofeedback program on the portable device, wherein the real-time brain electrical activity of the user is continuously displayed on the portable device as visual feedback and the user is able to control the brain electrical activity such that the visual feedback can be manipulated by the user in real-time; and
completing the neurofeedback program.

18. The method of claim 17 wherein the portable device includes a display screen for providing the visual feedback.

19. The method of claim 17 wherein the neurotherapy is used to improve a number of conditions and disorders selected from the group consisting of attention-deficit/hyperactivity disorder, memory problems, learning disorder, focusing disorder, sleep disorder, tourette's syndrome, pre-menstrual syndrome, migraines, seizures, extreme stress, anxiety attacks, autism and depression.

20. The method of claim 17, wherein the neurotherapy is used for peak performance training.
